(19) United States
(12) Patent Application Publication
(10) Pub. No.: US 2011/0182773 A1
(43) Pub. Date: Jul. 28, 2011

(54) Method and System for Controlling Microbiological Contamination

(76) Inventor: Alton R. Holt, Silsbee, TX (US)

(21) Appl. No.: 12/693,933

(22) Filed: Jan. 26, 2010

(51) Int. Cl. A61L 2/20; B01J 19/12

(52) U.S. Cl. 422/29; 422/186

(57) ABSTRACT

A system and method control microbes on a target. In one embodiment, the system controls microbes on a target with hydrogen peroxide gas. The system includes a purification device. The purification device includes a catalyst having titanium dioxide and a light disposed to emit electromagnetic radiation into the catalyst. The purification device is disposed to allow air to flow into the purification device and contact the catalyst, with a reaction producing hydrogen peroxide gas. The system also includes the hydrogen peroxide gas exiting the purification device. The hydrogen peroxide gas controls microbes on the target by degrading the microbes.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

This invention relates to the field of antimicrobials and more specifically to the field of controlling microbial contamination on specific targets using hydrogen peroxide gas as an antimicrobial.

2. Background of the Invention

There is an increasing need for disinfecting and preventing infection of food stocks. Such infection typically involves bacteria, viruses, mold, and the like. Conventional prevention methods include refrigeration. Conventional disinfection processes involve the application of detergents and liquid sanitizers. Drawbacks to such conventional methods include inefficiencies with applying refrigeration in certain locations (i.e., remote locations). Further drawbacks include inefficiencies in the frequency of the disinfection. For instance, such conventional disinfection processes are typically carried out on a daily basis or intermittently during a day. Additional drawbacks include the typical need to wash the food stocks of the detergents and liquid sanitizers prior to use by a consumer.

Consequently, there is a need for an improved antimicrobial system for protecting and removing microbiological contamination of food stocks.

### BRIEF SUMMARY OF SOME OF THE PREFERRED EMBODIMENTS

These and other needs in the art are addressed in one embodiment by a system that controls microbes on a target with hydrogen peroxide gas. The system includes a purification device. The purification device includes a catalyst having titanium dioxide and a light disposed to emit electromagnetic radiation into the catalyst. The purification device is disposed to allow air to flow into the purification device and contact the catalyst, with a reaction producing hydrogen peroxide gas. The system also includes the hydrogen peroxide gas exiting the purification device. The hydrogen peroxide gas controls microbes on the target by degrading the microbes.

These and other needs in the art are addressed in another embodiment by a method for controlling microbes on a target with a hydrogen peroxide gas. The method includes treating air in a purification device to produce hydrogen peroxide gas. The purification device includes a catalyst having titanium dioxide and a light disposed to emit electromagnetic radiation into the catalyst. The purification device is disposed to allow air to flow into the purification device and contact the catalyst, with a reaction producing the hydrogen peroxide gas. Treated air including the hydrogen peroxide gas exits the purification device. The method also includes directing the treated air to the target. In addition, the method includes controlling the microbes on the target with the hydrogen peroxide gas.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other embodiments for carrying out the same purposes of the present invention. It should also be realized by those skilled in the art that such equivalent embodiments do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates a microbe control system having a purification device, distributor, and a conveyance apparatus;

FIG. 2 illustrates a purification device;

FIG. 3 illustrates a catalyst; and

FIG. 4 illustrates a microbe control system having a purification device and a conveyance apparatus.

### DETAILED DESCRIPTION OF THE PREFERRED EMBODIMENTS

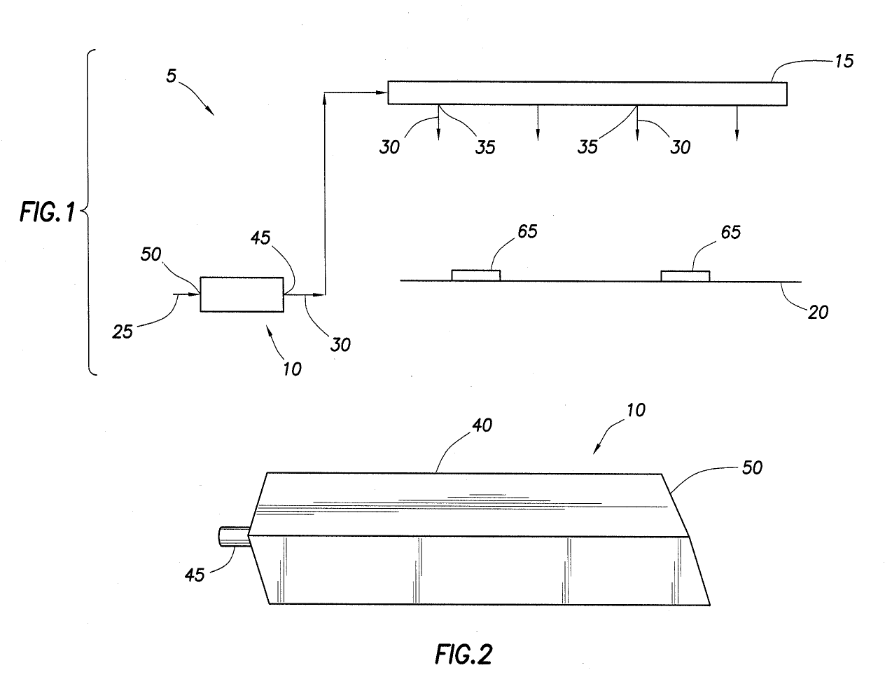

FIG. 1 illustrates an embodiment of microbe control system 5 having a purification device 10. Microbe control system 5 creates hydrogen peroxide gas and supplies the hydrogen peroxide gas to distributor 15 to control microbiological contaminants on targets 65. The microbiological contaminants include any type of microbe. In an embodiment, the microbes comprise fungi, mold, viruses, bacteria, or any combinations thereof. Microbe control system 5 controls the microbiological contaminants by degrading all or a portion of the microbiological contaminants on targets 65 (i.e., by killing the microbiological contaminants).

In an embodiment as illustrated in FIG. 1, purification device 10 takes in air 25 and produces treated air 30 including the produced hydrogen peroxide gas. In an embodiment, a blower (not illustrated) provides the air 25 to purification device 10. Purification devices are disclosed in U.S. application Ser. No. ______, filed on ______, 2010 and entitled “Microbe Reduction and Purification” with attorney docket number 1820-00100. U.S. application Ser. No. ______ is hereby incorporated by reference in its entirety.

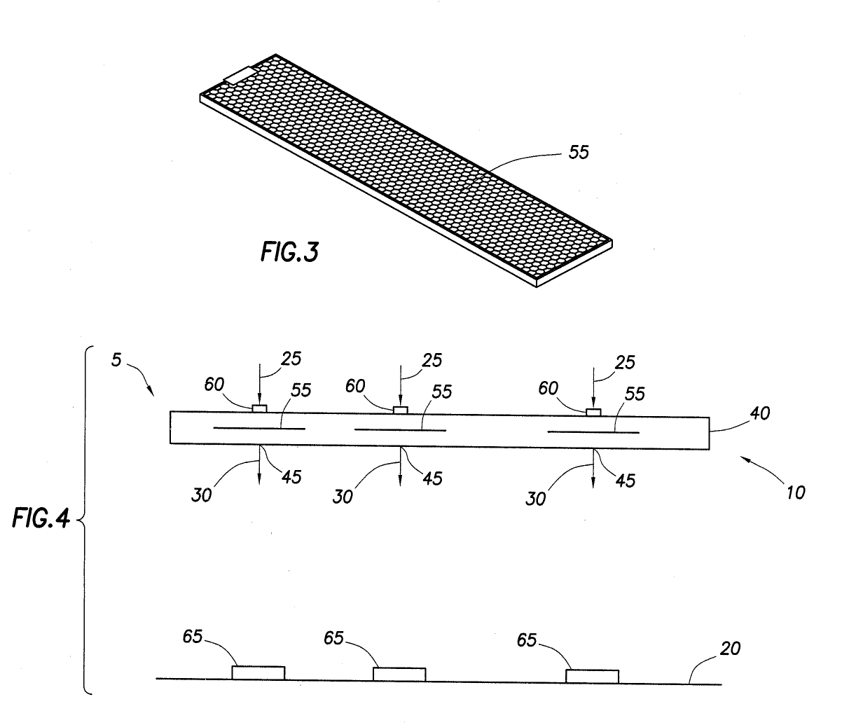

FIG. 2 illustrates an embodiment of purification device 10 having body 40, air inlet 50, gas outlet 45, and catalyst 55 (not illustrated). Air inlet 50 allows air to flow into purification device 10 and contact catalyst 55, which is disposed within the interior of purification device 10. In an embodiment, air 25 may be ambient air. Purification device 10 may include any suitable number of catalysts 55. FIG. 3 illustrates a front view of an embodiment of catalyst 55. Catalyst 55 comprises titanium dioxide. In other embodiments, catalyst 55 comprises titanium dioxide and metallic additives. Any metallic additives suitable for improving the reaction to produce the hydrogen peroxide gas may be used. In an embodiment, the metallic additives include copper, silver, rhodium, or any combinations thereof. Catalyst 55 may have any suitable configuration for use in purification device 10. In embodiments, catalyst 55 comprises a configuration of a plurality of cells. In an embodiment as illustrated in FIG. 3, catalyst 55 comprises a configuration of a plurality of hexagonal, walled cells (i.e., honeycomb shape configuration). Without being limited by theory, the hexagonal, walled cell configuration facilitates the reaction to produce the hydrogen peroxide because it provides an increased surface area for the reaction. The embodiment of catalyst 55 shown in FIG. 3 has a rectangular shape, but it is to be understood that catalyst 55 is not limited to a rectangular shape. In alternative embodiments, catalyst 55 may have any other suitable shape such as a square shape, triangular shape, and the like. In some embodiments (not illustrated), catalyst 55 is disposed inside purification device 10 at an angle in relation to the direction at which the air 25 flows into purification device 10 and contacts catalyst 55. In embodiments, catalyst 55 is disposed at angle in relation to the direction at which the air 25 flows into purification device 10 and contacts catalyst 55 with the angle between about 15 degrees and about 75 degrees, and alternatively at about 45 degrees. Without being limited by theory, disposing catalyst 55 at an angle to the direction at which the air 25 flows into purification device 10 increases the surface area available for the reaction to produce the hydrogen peroxide. For instance, as light and air 25 pass through catalyst 55, the catalyst 55 disposed at an angle increases the amount of contact of the light and air 25 with the surface of catalyst 55. Catalyst 55 includes a light (not illustrated) disposed inside the catalyst 55. The light is a non-ozone producing ultraviolet light. In embodiments, the light is a crystal ultraviolet light. Without limitation, commercial examples of non-ozone producing ultraviolet lights include the non ozone bulb provided by LightTech. Catalyst 55 includes one light. In alternative embodiments, catalyst 55 includes more than one light. The light is disposed to emit electromagnetic radiation into catalyst 55. For instance, the light emits electromagnetic radiation into the hexagonal, walled cells of catalyst 55 with the electromagnetic radiation contacting the surface of the cells.

FIG. 1 illustrates an embodiment of operation of microbe control system 5 with targets 65 disposed on conveyance apparatus 20. Targets 65 may be any target that is desired to be treated with treated air 30 comprising the produced hydrogen peroxide gas. For instance, targets 65 may include food stock, artifacts, paper goods, clothing, people, animals, plants, and the like. Conveyance apparatus 20 may include any type of device suitable for conveying targets 65. In embodiments, conveyance apparatus 20 is a conveyor belt system. In an embodiment as illustrated, purification device 10 takes in air 25 through air inlet 50. Air 25 flows into purification device 10 and contacts catalyst 55 with the air 25 passing through catalyst 55. It is to be understood that the air (i.e., ambient air) has a moisture content and is comprised of water vapor and oxygen. Catalyst 55 and the moisture in the ambient air (i.e., the water vapor and oxygen) are exposed to the electromagnetic radiation from the light. A reaction between the titanium dioxide, the moisture in the ambient air, and the electromagnetic radiation produce the hydrogen peroxide gas. In an embodiment, the reaction is a photo-catalytic reaction. For instance, in embodiments, moisture from the ambient air contacts catalyst 55 as it flows through catalyst 55. The electromagnetic radiation from the light contacts the various surfaces of catalyst 55 and reacts with the moisture against the titanium dioxide to produce the hydrogen peroxide gas. The reaction in purification device 10 to produce the hydrogen peroxide gas does not produce ozone.

Without being limited by theory, the produced hydrogen peroxide gas has both positive and negative charges. With such charges, the hydrogen peroxide gas is drawn to microbes by electrostatic attraction. For instance, the hydrogen peroxide gas is drawn to the positive and negative charges of the surface of the microbes. The hydrogen peroxide gas then controls the microbes by chemically degrading the microbes, which may be degraded cell by cell. In embodiments in which the microbes are attached to targets 65 such as paper, the hydrogen peroxide gas degrades the microbes down to the point of attachment. In some instances, the microbes release from the surface and may be removed. In embodiments, the microbes may be removed without removing structurally sound material. The hydrogen peroxide gas also diffuses into porous material (i.e., anywhere that air flows) such a paper and cloth, which allows degradation of the microbes in the paper and cloth.

As illustrated in FIG. 1, treated air 30 includes the hydrogen peroxide gas produced from purification device 10. Treated air 30 exits purification device 10 through gas outlet 45 and flows to distributor 15. In embodiments, treated air 30 flows through piping (not illustrated) to distributor 15. Distributor 15 may be any type of distribution apparatus suitable for directing the flow of treated air 30 to targets 65. In an embodiment as illustrated in FIG. 1, distributor 15 is a pipe with perforations 35 on bottom side 70 of distributor 15. Distributor 15 may have any desired number of perforations 35 suitable for applying treated air 30 to targets 65. Distributor 15 may have any length suitable for applying treated air 30 to targets 65. Distributor 15 is not limited to piping but may have any other configuration suitable for applying treated air 30 to targets 65. Treated air 30 flows into distributor 15 and flows out of bottom side 70 through perforations 35. Distributor 15 is disposed to allow treated air 30 to exit through perforations 35 in a direction at which treated air 30 is directed to contact targets 65. In some embodiments as illustrated, distributor 15 is about parallel to conveyance apparatus 20. Treated air 30 exits distributor 15 and then contacts targets 65. The hydrogen peroxide gas in treated air 30 controls microbes on and/or in targets 65. Conveyance apparatus 20 moves targets 65 underneath distributor 15. In alternative embodiments (not illustrated), microbe control system 5 does not include conveyance apparatus 20 but instead has targets 65 disposed underneath distributor 15 for contact with the exiting hydrogen peroxide gas.

FIG. 4 illustrates an embodiment of microbe control system 5 having another embodiment of purification device 10. As illustrated, purification device 10 has fans 60. The fans 60 take air 25 from outside purification device 10 and provide such air 25 to catalyst 55. In embodiments as shown, fans 60 are disposed on the opposing side of catalyst 55 from gas outlet 45. Purification device 10 may have any desirable number of fans 60. In some embodiments, purification device 10 has one fan 60 for each catalyst 55. The fans 60 may be any fan of a power suitable for providing air 25 through catalyst 55 and then providing sufficient air pressure in purification device 10 to force the produced hydrogen peroxide out of purification device 10. In such embodiment, purification device 10 may have any desired number of gas outlets 45 suitable for applying treated air 30 to targets 65. Purification device 10 may have any length suitable for applying treated air 30 to targets 65. In an embodiment as shown in FIG. 4, purification device 10 is a tube. It is to be understood that purification device 10 is not limited to tubing but may have any other configuration suitable for producing hydrogen peroxide gas and applying treated air 30 to targets 65. In some embodiments as illustrated, distributor 15 is about parallel to conveyance apparatus 20. Treated air 30 exits distributor 15 and then contacts targets 65. The hydrogen peroxide gas in treated air 30 controls microbes on and/or in targets 65. Conveyance apparatus 20 moves targets 65 underneath distributor 15. In alternative embodiments (not illustrated), microbe control system 5 does not include conveyance apparatus 20 but instead has targets 65 disposed underneath distributor 15 for contact with the exiting hydrogen peroxide gas.

In operation of an embodiment as illustrated in FIG. 4, fans 60 operate to take air 25 into purification device 10. Air 25 flows into purification device 10 and contacts catalysts 55 with the air 25 passing through catalysts 55. Catalysts 55 and the moisture in the ambient air (i.e., the water vapor and oxygen) are exposed to the electromagnetic radiation from the light and produce the hydrogen peroxide gas. Treated air 30 includes the hydrogen peroxide gas produced from purification device 10. Treated air 30 exits purification device 10 through gas outlet 45 in a direction at which treated air 30 is directed to contact targets 65. The hydrogen peroxide gas in treated air 30 controls microbes on and/or in targets 65. Conveyance apparatus 20 moves targets 65 underneath distributor 15. In alternative embodiments (not illustrated), microbe control system 5 does not include conveyance apparatus 20 but instead has targets 65 disposed underneath distributor 15 for contact with the exiting hydrogen peroxide gas. For instance, in such alternative embodiments, microbe control system 5 may be disposed in a container, vehicle or the like, and targets 65 may be people or animals exposed to microbes. Targets 65 may be disposed underneath the purification device 10, and the produced hydrogen peroxide gas controls the microbes on targets 65 and also on any clothing or other materials on targets 65.

Although the present invention and its advantages have been described in detail, it should be understood that various changes, substitutions and alterations may be made herein without departing from the spirit and scope of the invention as defined by the appended claims.

What is claimed is:

1. A system for controlling microbes on a target with hydrogen peroxide gas, comprising:
a purification device, wherein the purification device comprises a catalyst comprising titanium dioxide and a light disposed to emit electromagnetic radiation into the catalyst, and wherein the purification device is disposed to allow air to flow into the purification device and contact the catalyst, with a reaction producing the hydrogen peroxide gas;
wherein the hydrogen peroxide gas exits the purification device, and wherein the hydrogen peroxide gas controls microbes on the target by degrading the microbes.

2. The system of claim 1, wherein the catalyst comprises a plurality of hexagonal, walled cells.

3. The system of claim 1, wherein the light comprises a non-ozone producing ultraviolet light.

4. The system of claim 1, wherein the catalyst is disposed to provide a reaction surface by which the air reacts when exposed to the catalyst and the electromagnetic radiation.

5. The system of claim 1, further comprising a distributor.

6. The system of claim 5, wherein the hydrogen peroxide gas flows from the purification device to the distributor, and wherein the distributor is adapted to allow the hydrogen peroxide gas to exit the distributor and contact the target.

7. The system of claim 5, wherein the distributor comprises a perforation disposed on a bottom side of the distributor.

8. The system of claim 1, wherein the purification device comprises a fan.

9. The system of claim 8, wherein the purification device comprises a gas outlet, and wherein the fan is disposed on an opposing side of the catalyst from the gas outlet.

10. The system of claim 1, wherein the purification device comprises more than one catalyst, and wherein each catalyst has a fan that blows air through the each catalyst.

11. A method for controlling microbes on a target with a hydrogen peroxide gas, comprising:
(A) treating air in a purification device to produce hydrogen peroxide gas, wherein the purification device comprises a catalyst comprising titanium dioxide and a light disposed to emit electromagnetic radiation into the catalyst, and further wherein the purification device is disposed to allow air to flow into the purification device and contact the catalyst, with a reaction producing the hydrogen peroxide gas, and wherein treated air comprising the hydrogen peroxide gas exits the purification device;
(B) directing the treated air to the target; and
(C) controlling the microbes on the target with the hydrogen peroxide gas.

12. The method of claim 11, wherein the catalyst comprises a plurality of hexagonal, walled cells.

13. The method of claim 11, wherein the light comprises a non-ozone producing ultraviolet light.

14. The method of claim 11, wherein the catalyst is disposed to provide a reaction surface by which the air reacts when exposed to the catalyst and the electromagnetic radiation.

15. The method of claim 11, wherein directing the treated air comprises a distributor directing the treated air.

16. The method of claim 15, wherein the hydrogen peroxide gas flows from the purification device to the distributor, and wherein the distributor is adapted to allow the hydrogen peroxide gas to exit the distributor and contact the target.

17. The method of claim 16, wherein the distributor comprises a perforation disposed on a bottom side of the distributor.

18. The method of claim 11, further comprising blowing the air through the catalyst.

19. The method of claim 18, wherein the purification device comprises a gas outlet and a fan, and wherein the fan is disposed on an opposing side of the catalyst from the gas outlet.

20. The method of claim 11, wherein the purification device comprises more than one catalyst, and wherein each catalyst has a fan that blows air through the each catalyst.
